(19) United States
(12) Patent Application Publication
(10) Pub. No.: US 2005/0014940 A1
(43) Pub. Date: Jan. 20, 2005

(54) Process for the preparation of 2-or-9-oxa-3, 7-diazabicyclo (3.3.1) nonanes from 2-aminomethyl-2, 3-dihydrooxazines intermediates therefore, and processes for preparing such intermediates

(76) Inventor: Gill Duncan Michael, Loughborough (GB)

Correspondence Address: NIXON & VANDERHYE, PC, 1100 N GLEBE ROAD, 8TH FLOOR, ARLINGTON, VA, 22201-4714, US

(21) Appl. No.: 10/496,498

(22) PCT Filed: Nov. 20, 2002
(86) PCT No.: PCT/GB02/05262

(30) Foreign Application Priority Data
Nov. 22, 2001 (SE) 0103908-0

(51) Int. Cl. C07D 498/04

(52) U.S. Cl. 544/74

(57) ABSTRACT

There is provided a process for the preparation of a compound of formula (I), which process comprises reaction of a compound(of formula (II), with either: (a) a formaldehyde and a compound of formula (III), R^{a}—OH and/or (b) a protected derivative of formaldehyde, wherein R¹, R² and R^{a} have meanings given in the description.

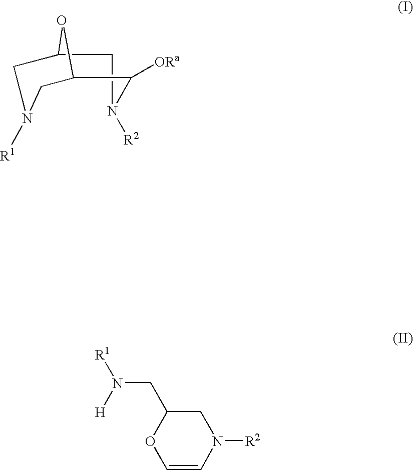

### FIELD OF THE INVENTION

This invention relates to a novel process for the preparation of oxabispidine compounds.

### BACKGROUND

The number of documented compounds including the 9-oxa-3,7-diazabicyclo-[3.3.1]nonane (oxabispidine) structure is very few. As a result, there are very few known processes that are specifically adapted for the preparation of oxabispidine compounds.

Certain oxabispidine compounds are disclosed in Chem. Ber. 96(11), 2827 (1963) as intermediates in the synthesis of 1,3diaza-6-oxa-adamantanes.

Hemiacetals (and related compounds) having the oxabispidine ring structure are disclosed in J. Org. Chem 31, 277 (1966), ibid 61(25), 8897 (1996), ibid 63(5), 1566 (1998) and ibid. 64(3), 960 (1999) as unexpected products from the oxidation of 1,5-diazacyclooctane-1,3-diols or the reduction of 1,5-diazacyclooctane-1,3-diones.

1,3-Dimethyl-3,7-ditosyl-9-oxa-3,7azabicyclo[3.3.1]nonane is disclosed in J. Org. Chem 32, 2425 (1967) as a product from the attempted acetylation of trans-1,3-dimethyl-1,5-ditosyl-1,5-diazacyclooctane-1,3-diol.

International patent application WO 01/28992 describes the synthesis of a wide range of oxabispidine compounds, which compounds are indicated as being useful in the treatment of cardiac arrhythmias. In WO 01/28992, processes for the formation of the oxabispidine ring system are disclosed, which processes all involve the formation of oxabispidine precursors in mixtures of cis and trans isomers. Such processes have the disadvantage that only one of those two isomers may be reacted to give the desired oxabispidine ring system.

None of the above-mentioned documents disclose or suggest the synthesis of oxabispidine compounds via 2,3-dihydrooxazines. We have now found, surprisingly, that oxabispidine compounds may be prepared conveniently by way of the cyclisation of 2-aminomethyl-substituted 2,3-dihydrooxazines.

### DISCLOSURE OF THE INVENTION

According to a first aspect of the invention there is provided a process for the preparation of a compound of formula I, 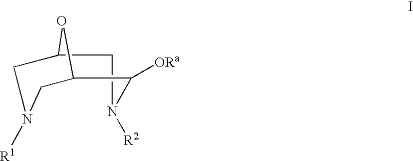
wherein
R¹ represents H, aryl or a structural fragment of formula Ia, 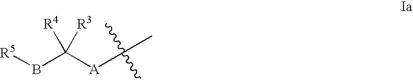
   in which
R³ represents H, halo, C₁₋₆ alkyl, —OR⁶, -E-N(R⁷)R⁸ or, together with R⁴, represents ═O;
R⁴ represents H, C₁₋₆ alkyl or, together with R³, represents ═O;
R⁶ represents H, C₁₋₆ alkyl, -E-aryl, -E-Het¹, —C(O)R^{9a}, —C(O)OR^{9b} or —C(O)N(R^{10a})R^{10b};
R⁷ represents H, C₁₋₆ alkyl, -E-aryl, -E-Het¹, —C(O)R^{9a}, —C(O)OR^{9b}, —S(O)₂R^{9c}, —[C(O)]ₚN(R^{10a})R^{10b} or —C(NH)NH₂;
R⁸ represents H, C₁₋₆ alkyl, -E-aryl or —C(O)R^{9d};
R^{9a} to R^{9d} independently represent, at each occurrence,
C₁₋₆ alkyl (optionally substituted and/or terminated by one or more substituents selected from halo, aryl and Het²), aryl, Het, or R^{9a} and R^{9d} independently represent H;
R^{10a} a R^{10b} independently represent, at each occurrence, H or C₁₋₆ alkyl (optionally substituted and/or terminated by one or more substituents selected from halo, aryl and Het⁴), aryl, Het⁵, or together represent C₃₋₆ alkylene, optionally interrupted by an O atom;
E represents, at each occurrence, a direct bond or C₁₋₄ alkylene;
p represents 1 or 2;
A represents -G-, -J-N(R¹¹)— or -J-O— (in which latter two groups, N(R¹¹)— or O— is attached to the carbon atom bearing R³ and R⁴);
B represents -Z-, -Z-N(R¹²)—, —NR¹²)-Z-, -Z-S(O)ₙ— or -Z-O— (in which latter two groups, Z is attached to the carbon atom bearing R³ and R⁴);
G represents a direct bond or C₁₋₆ alkylene;
J represents C₂₋₆ alkylene;
Z represents a direct bond or C₁₋₄ alkylene;
R¹¹ and R¹² independently represent H or C₁₋₆ alkyl;
n represents 0, 1 or 2;
R⁵ represents aryl or heteroaryl, both of which groups are optionally substituted by one or more substituents selected from —OH, cyano, halo, nitro, C₁₋₆ alkyl (optionally terminated by —N(H)C(O)OR^{13a}), C₁₋₆ alkoxy,
—N(R^{14a})R^{14b}, —C(O)R^{14c}, —C(O)OR^{14d}, —C(O)N(R^{14e})R^{14f}, —N(R^{14g})C(O)(R^{14h},
—N(R¹⁴ⁱ)C(O)N(R^{14j})R^{14k}, —N(R^{14m})S(O)₂R^{13b}, —S(O)₂R^{13c} and/or
—OS(O)₂R^{13d};
R^{13a} to R^{13d} independently represent C₁₋₆ alkyl;
R^{14a} and R^{14b} independently represent H, C₁₋₆ alkyl or together represent C₃₋₆ alkylene, resulting in a four- to seven-membered nitrogen-containing ring;
R^{14c} to R^{14m} independently represent H or C₁₋₆ alkyl;
Het¹ to Het⁵ independently represent, at each occurrence, five- to twelve-membered heteroaryl groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{15a})R^{15b}, —C(O)R^{15c}, —C(O)OR^{15d}, —C(O)N(R^{15e})R^{15f}, —N(R^{15g})C(O)R^{15h} and —N(¹⁵ⁱ)S(O)₂R^{15j};
R^{15a} to R^{15j} independently represent C₁₋₆ alkyl, aryl or R^{15a} to R¹⁵ⁱ independently represent H; and
wherein each aryl and aryloxy group, unless otherwise specified, is optionally substituted; provided that:
(a) when R⁴ represents H or C₁₋₄ alkyl; and
   A represents -J-N(R¹¹) or -J-O—;
   then B does not represent —N(R¹²)—, —S(O)ₙ—, —O— or —N(R¹²)-Z- (in which latter group —N(R¹²) is attached to the carbon atom bearing R³ and R⁴);
(b) when R³ represents —OR⁶ or -E-N(R⁷)R⁸ in which E represents a direct bond, then:
   (i) A does not represent a direct bond, -J-N(R¹¹)— or -J-O—; and
   (ii) B does not represent —N(R¹²)—, —S(O)ₙ—, —O— or —N(R¹²)-Z- (in which latter group —N(R¹²) is attached to the carbon atom bearing R³ and R⁴);
(c) when A represents a direct bond, then R³ and R⁴ do not together represent ═O;
R² represents an electron withdrawing amino protecting group; and
R^{a} represents C₁₋₄ alkyl or benzyl,
which process comprises reaction of a compound of formula II, 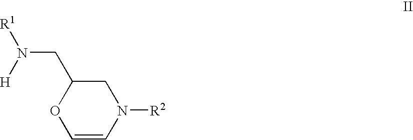
   wherein R¹ and R² are as defined above, with either:
(a) a formaldehyde and a compound of formula III,
   R^{a}—OH III
   wherein R^{a} is as defined above; and/or
(b) a protected derivative of a formaldehyde,
which process is referred to hereinafter as “the process of the invention”.

A preferred process of the invention involves the reaction of a compound of formula II as hereinbefore defined with a formaldehyde and a compound of formula III as hereinbefore defined.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

Unless otherwise specified, alkylene groups as defined herein may be straight-chain or, when there is a sufficient number (i.e. a minimum of two) of carbon atoms, be branched-chain. Such alkylene chains may also be saturated or, when there is a sufficient number (i.e. a minimum of two) of carbon atoms, be unsaturated and/or interrupted by one or more oxygen and/or sulfur atoms. Unless otherwise specified, alkylene groups may also be substituted by one or more halo (e.g. fluoro) atoms.

The term “aryl”, when used herein, includes C₆₋₁₀ aryl groups such as phenyl, naphthyl and the like. The term “aryloxy”, when used herein includes C₆₋₁₀ aryloxy groups such as phenoxy, naphthoxy and the like. For the avoidance of doubt, aryloxy groups referred to herein are attached to the rest of the molecule via the O-atom of the oxy-group. Unless otherwise specified, aryl and aryloxy groups may be substituted by one or more substituents including —OH, cyano, halo, nitro, C₁₋₆ alkyl, C₁₋₆ alkoxy, —N(R^{14a})R^{14b}, —C(O)R^{14c}, —C(O)OR^{14d}, —C(O)N(R^{14e})R^{14f}, —N(R^{14g})C(O)R^{14h}, —N(R^{14m})S(O)₂R^{13b}, —S(O)₂R^{13c} and/or —OS(O)₂R^{13d} (wherein R^{13b} to R^{13d} and R^{14a} to R^{14m} are as hereinbefore defined). When substituted, aryl and aryloxy groups are preferably substituted by between one and three substitutents.

Heteroaryl groups that may be mentioned include those containing 1 to 4 heteroatoms (selected from the group oxygen, nitrogen and/or sulfur) and in which the total number of atoms in the ring system are between five and twelve. Heteroaryl groups may be fully saturated, wholly aromatic, partly aromatic and/or bicyclic in character. Heteroaryl groups that may be mentioned include benzodioxanyl, benzodioxepanyl, benzodioxolyl, benzofuranyl, benzimidazolyl, benzomorpholinyl, benzoxazinonyl, benzothiophenyl, chromanyl, cinnolinyl, dioxanyl, furanyl, imidazolyl, imidazo[1,2-a]pyridinyl, indolyl, isoquinolinyl, isoxazolyl, morpholinyl, oxazolyl, phthalazinyl, piperazinyl, piperidinyl, purinyl, pyranyl, pyrazinyl, pyrazolyl, pyridinyl, pyrimindinyl, pyrrolidinonyl, pyrrolidinyl, pyrrolinyl, pyrrolyl, quinazolinyl, quinolinyl, tetrahydropyranyl, tetrahydrofuiranyl, thiazolyl, thienyl, thiochromanyl, triazolyl and the like. Substituents on heteroaryl groups may, where appropriate, be located on any atom in the ring system including a heteroatom. The point of attachment of heteroaryl groups to the rest of the molecule may be via any atom in the ring system including (where appropriate) a heteroatom, or an atom on any fused carbocyclic ring that may be present as part of the ring system. Heteroaryl groups may also be in the N- or S-oxidised form. When R⁵ is a heteroaryl group, preferred heteroaryl groups include pyridinyl groups.

The term “halo”, when used herein, includes fluoro, chloro, bromo and iodo.

As used herein, the term “amino protecting group” includes groups mentioned in “Protective Groups in Organic Synthesis”, 2^{nd} edition, T W Greene & P G M Wutz, Wiley-Interscience (1991), in particular those indexed at the start of the chapter entitled “Protection for the Amino Group” (see pages 309 to 315) of that reference, the disclosure in which document is hereby incorporated by reference.

Specific examples of amino protecting groups thus include:
(a) those which form carbamate groups (e.g. to provide methyl, cyclopropylmethyl, 1-methyl-1-cyclopropylmethyl, diisopropyl-methyl, 9-fluorenylmethyl, 9-(2-sulfo)fluorenylmethyl, 2-furanylmethyl, 2,2,2-trichloroethyl, 2-haloethyl, 2-trimethylsilylethyl, 2-methylthioethyl, 2-methyl-sulfonylethyl, 2(p-toluenesulfonyl)ethyl, 2-phosphonioethyl, 1,1-dimethylpropynyl, 1,1-dimethyl-3-(N,N-dimethylcarboxamido)propyl, 1,1-dimethyl-3-(N,N-diethylamino)-propyl, 1-methyl-1-(1-adamantyl)ethyl, 1-methyl-1-phenylethyl, 1-methyl-1-(3,5-di-methoxyphenyl)ethyl, 1-methyl-1-(4-biphenylyl)-ethyl, 1-methyl-1-(p-phenylazophenyl)ethyl, 1,1-dimethyl-2-haloethyl, 1,1-dimethyl-2,2,2-trichloroethyl, 1,1-dimethyl-2-cyanoethyl, isobutyl, t-butyl, t-amyl, cyclobutyl, 1-methylcyclobutyl, cyclopentyl, cyclohexyl, 1-methylcyclohexyl, 1-adamantyl, isobornyl, vinyl, allyl, cinnamyl, phenyl, 2,4,6-tri-t-butylphenyl, m-nitrophenyl, S-phenyl, 8-quinolinyl, N-hydroxypiperidinyl, 4-(1,4-dimethylpiperidinyl), 4,5-diphenyl-3-oxazolin-2-one, benzyl, 2,4,6-methylbenzyl, p-methoxybenzyl, 3,5-dimethoxybenzyl,p-decyloxybenzyl,p-nitro-benzyl, o-nitrobenzyl, 3,4-dimethoxy-6-nitrobenzyl,p-bromobenzyl, chlorobenzyl, 2,4-dichloro-benzyl,p-cyanobenzyl, o-(N,N-dimethyl-carboxamidobenzyl)benzyl, m-chloro-p-acyloxybenzyl, p-(dihydroxy-boryl)benzyl, p-(phenylazo)benzyl, p-(p′-methoxyphenylazo)benzyl, 5-benzisoxazolylmethyl, 9-anthrylmethyl, diphenylmethyl, phenyl(o-nitrophenyl)methyl, di(2-pyridyl)methyl, 1-methyl-1-(4-pyridyl)-ethyl, isonicotinyl, or S-benzyl, carbamate groups);
(b) those which form amide groups (e.g. to provide N-formyl, N-acetyl, N-chloroacetyl, N-dichloroacetyl, N-trichloroacetyl, N-trifluoroacetyl, N-o-nitrophenylacetyl, N-o-nitrophenoxyacetyl, N-acetoacetyl, N-acetylpyridinium, N-3-phenylpropionyl, N-3-(p-hydroxyphenyl)-propionyl, N-3-(o-nitrophenyl)propionyl, N-2-methyl-2-(o-nitrophen-oxy)propionyl, N-2-methyl-2-(o-phenylazophenoxy)propionyl, N4-chlorobutyryl, N-isobutyryl, N-o-nitrocinnamoyl, N-picolinoyl, N-(N′-acetylmethionyl), N-(N′-benzoylphenylalanyl), N-benzoyl, N-p-phenylbenzoyl, N-p-methoxybenzoyl, N-o-nitrobenzoyl, or N-o-(benzoyloxymethyl)benzoyl, amide groups);
(c) alkyl groups (e.g. N-allyl, N-phenacyl, N-3-acetoxypropyl, N-(4-nitro-1-cyclohexyl-2-oxo-pyrrolin-3-yl), N-methoxymethyl, N-chloroethoxymethyl, N-benzyloxymethyl, N-pivaloyloxymethyl, N-2-tetrahydropyranyl, N-2,4-dinitrophenyl, N-benzyl, N-3,4-di-methoxybenzyl, N-o-nitrobenzyl, N-di(p-methoxyphenyl)methyl, N-triphenylmethyl, N-(p-methoxyphenyl)diphenylmethyl, N-diphenyl-4-pyridylmethyl, N-2-picolyl N′-oxide or N-dibenzosuberyl groups);
(d) phosphinyl and phosphoryl groups (e.g. N-diphenylphosphinyl, N-dimethylthiophosphinyl, N-diphenylthiophosphinyl, N-diethyl-phosphoryl, N-dibenzylphosphoryl or N-phenylphosphoryl groups);
(e) sulfenyl groups (e.g. N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl or N-triphenylmethylsulfenyl groups);
(f) sulfonyl groups (e.g. N-benzenesulfonyl, N-p-methoxybenzene-sulfonyl, N-2,4,6-trimethylbenzenesulfonyl, N-toluenesulfonyl, N-benzylsulfonyl, N-p-methylbenzylsulfonyl, N-trifluoromethylsulfonyl or N-phenacylsulfonyl); and
(g) the N-trimethylsilyl group.

Electron-withdrawing amino protecting groups include the sulfonyl groups mentioned above, as well as those which form amide or, particularly, carbamate groups mentioned above, such as tert-butoxycarbonyl (to provide a tert-butyl carbamate group) and, particularly, benzyloxycarbonyl (to provide a benzyl carbamate group).

The skilled person will also appreciate that certain values of the structural fragment of formula Ia may also be referred to as amino protecting groups (see e.g. group (c) in the list above).

Suitable formaldehydes for use in the process of the invention include paraformaldehyde. Suitable protected derivatives of formaldehyde include those that are protected at the carbonyl group (e.g. as C₁₋₄ alkyl acetals, such as methyl acetals) and that react with the compound of formula II to give an intermediate that is capable of undergoing cyclisation to give a compound of formula I.

Preferred values of R¹ include H or structural fragments of formula Ia in which:
R³ represents H, methyl, —OR⁶ or —N(H)R⁷;
R⁴ represents H or methyl;
R⁶ represents H, C₁₋₂ alkyl or phenyl (which phenyl group is optionally substituted by one or more substituents selected from cyano and C₁₋₄ alkoxy);
R⁷ represents H, C₁₋₂ alkyl, phenyl (which phenyl group is optionally substituted by one or more substituents selected from cyano, halo, nitro, C₁₋₄ alkyl and C₁₋₄ alkoxy), —C(O)R^{9a} or —C(O)OR^{9b};
R^{9a} and R^{9b} independently represent C₁₋₆ alkyl;
A represents a direct bond or C₁₋₄ alkylene;
B represents -Z-, -Z-N(R¹²)—, -Z-S(O)₂— or -Z-O—;
R¹² represents H or methyl;
R⁵ represents pyridinyl or phenyl, which latter group is optionally substituted by one to three substituents selected from cyano, nitro, C₁₋₂ alkoxy, NH₂ and —N(H)S(O)₂CH₃.

More preferred values of R¹ include H or structural fragments of formula Ia in which:
R³ represents H, —OR⁶ or —N(H)R⁷;
R⁴ represents H;
R⁶ represents H or phenyl (optionally substituted by one or more substituents selected from cyano and C₁₋₂ alkoxy);
R⁷ represents H, phenyl (optionally substituted by one or more cyano groups) or —C(O)O—C₁₋₅ alkyl;
A represents a single bond or C₁₋₃ alkylene;
represents -Z-, -Z-N(H)—, -Z-S(O)₂— or -Z-O—;
R⁵ represents phenyl optionally substituted by cyano in the ortho- and/or, in particular, the para-position relative to B.

Particularly preferred values of R¹ include structural fragments of formula Ia in which:
R³ represents H;
A represents methylene, ethylene or, especially, a single bond;
B represents a single bond;
R⁵ represents unsubstituted phenyl.

Preferred values of R^{a} include ethyl and, particularly, methyl.

The process of the invention is preferably carried out under one or more of the following conditions.
(a) In the presence of a suitable solvent system. Suitable solvents include polar and/or hydroxylic solvents such as acetonitrile, C₁₋₄ alkyl alcohols, toluene and mixtures thereof.
(b) In the presence of a suitable catalyst (e.g. an acidic catalyst such as a Lewis acid or a Brönsted acid (e.g. a sulfonic acid such as p-toluenesulfonic acid)).
(c) At or above room temperature (e.g. from room temperature to the reflux temperature of the solvent system that is employed). When the solvent that is employed is a mixture of a C₁₋₄ alkyl alcohol (such as methanol) and acetonitrile, reaction is preferably carried out at reflux.
(d) Using one or more equivalents (relative to the compound of formula II) of the formaldehyde (and/or a suitable protected derivative thereof), for example between 1 and 10 equivalents (such as between 1 and 5 (e.g. between 2 and 4) equivalents).
(e) Using one or more equivalents (relative to the compound of formula II) of the compound of formula III (e.g. an excess such as 10 or more equivalents).
(f) By reacting the compound of formula II with one or more (e.g. three) equivalents of a formaldehyde (e.g. paraformaldehyde), in the presence of an excess of a compound of formula III.

The process of the invention is preferably carried out to provide compounds of formula I in which R¹ represents benzyl and R² represents benzyloxycarbonyl.

Compounds of formula II may be prepared by methods known to those skilled in the art. For example, compounds of formula II, and derivatives thereof, may be prepared by elimination of an alcohol from a corresponding 6-aminomethyl-substituted 2-alkoxymorpholine.

For example, compounds of formula II may be prepared by elimination of R^{b}OH from a compound of formula IV, 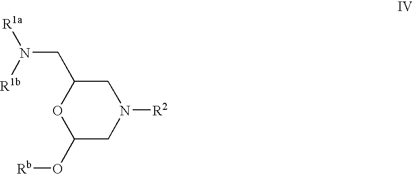
wherein
R^{1a} represents an aryl group, a structural fragment of formula Ia as hereinbefore defined, an electron withdrawing amino protecting group as hereinbefore defined, or, together with R^{1b}, represents a cyclic amino protecting group;
R^{1b} represents an electron withdrawing amino protecting group as hereinbefore defined, or, together with R^{1a}, represents a cyclic amino protecting group;
R^{b} represents C₁₋₄ alkyl; and
R² is as hereinbefore defined,
followed by deprotection (as necessary) of the nitrogen atom to which the groups R^{1a} and R^{1b} are attached and then, if necessary (i.e. in cases where the group R^{1a}, in the compound of formula IV formed by way of the elimination step, does not represent an aryl group or a structural fragment of formula Ia), reaction of the deprotected amine with a compound that provides the aryl group or the structural fragment of formula la as hereinbefore defined. The term “cyclic amino protecting group” will be understood by those skilled in the art to include all amino protecting groups that, when bound to the nitrogen atom of the amino group, form a cyclic system incorporating that nitrogen atom. The term therefore includes groups that form cyclic imido groups, such as succinimide and, particularly, phthalimide groups.

As before, the term “electron-withdrawing amino protecting group” includes those which form carbamate and amide groups, as well as phosphoryl and sulfonyl groups, mentioned hereinbefore in relation to the term “amino protecting group”. Preferred electron-withdrawing amino protecting groups that R² may represent in compounds of formula IV include those which form carbamate groups, e.g. benzyloxycarbonyl.

Preferred values of R^{b} include ethyl and, particularly, methyl.

It is preferred that elimination of R^{b}OH from a compound of formula IV (hereinafter referred to as the “elimination process”) is carried out on a compound of formula IV in which R^{1a} and R^{1b} together represent a cyclic amino protecting group. Preferred cyclic amino protecting groups thus include those which form cyclic imido groups such as phthalimide groups.

The elimination process is preferably carried out under one or more of the following conditions:
(a) In the presence of a suitable solvent system. Suitable solvents include those that are capable of facilitating elimination of R^{b}OH and yet will not react with the iminium ion intermediate, such as an aromatic hydrocarbon, e.g. toluene.
(b) In the presence of a suitable catalyst (e.g. an acidic catalyst such as a Lewis acid or a Brönsted acid (e.g. a sulfonic acid such asp-toluenesulfonic acid)).
(c) At elevated temperature (e.g. from above room temperature to around the reflux temperature of the solvent system that is employed). When the solvent that is employed is toluene, the reaction is preferably carried out at reflux.
(d) In the presence of an alcohol sorbing agent (e.g. molecular sieves (such as 3Å molecular sieves)).

Following the elimination process, deprotection of the nitrogen atom to which the groups R^{1a} and R^{1b} are attached (which may comprise removal of both R^{1a} and R^{1b}, or of R^{1b} alone) may be carried out by way of routine techniques. For example, when R^{1a} and R^{1b} together represents a cyclic amino protecting group, such as phthalimide, deprotection may be carried out by way of reaction with hydrazine, for example as described hereinafter.

When the deprotection is followed by reaction with a compound that provides the structural fragment of formula Ia, the latter transformation may be achieved using methods known to those skilled in the art, for example by analogy with coupling, and protection (e.g. in the case where the structural fragment Ia may be described as a protecting group, such as a benzyl group), methods disclosed in WO 01/28992. For example, this may be carried out by reaction of a compound of formula II in which R¹ represents H with a compound of formula V,
R⁵BC(R³)(R⁴)AL¹ V
wherein L¹ represents a suitable leaving group, such as halo, alkanesulfonate (e.g. mesylate), perfluoroalkanesulfonate or arenesulfonate (e.g. 2- or 4-nitrobenzenesulfonate, toluenesulfonate or benzenesulfonate) and A, B, R³, R⁴ and R⁵ are as hereinbefore defined, under reaction conditions that are well known to those skilled in the art, for example at elevated temperature (e.g. between 35° C. and reflux temperature) in the presence of a suitable base (e.g. triethylamine or potassium carbonate) and an appropriate organic solvent (e.g. acetonitrile, dichloromethane, chloroform, dimethylsulfoxide, N,N-dimethylformamide, a lower alkyl alcohol (e.g. ethanol), isopropyl acetate or mixtures thereof). In the case of compounds of formula II in which R¹ represents a benzyl group, such compounds may also be made by reaction of the deprotected amine with benzaldehyde, followed by reduction of the resultant intermediate (e.g. as described hereinafter).

Compounds of formula IV may be prepared by methods known to those skilled in the art. For example, compounds of formula IV, or derivatives thereof, may be prepared by cyclisation of compound of formula VI, 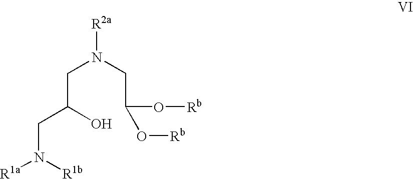
wherein
R^{2a} represents an amino protecting group as hereinbefore defined; and
R^{1a}, R^{1b} and R^{b} are as hereinbefore defined, followed by, if necessary (i.e. in cases where R^{2a} does not represent an electron-withdrawing amino protecting group as hereinbefore defined), replacement of the amino protecting group R^{2a} by an electron-withdrawing amino protecting group R².

Preferred values of R^{2a} include amino protecting groups mentioned hereinbefore and, particularly, alkylaryl groups such as C₁₋₃ alkylphenyl and especially benzyl. In this respect, the above cyclisation process is preferably carried out using a compound of formula VI in which R^{2a} represents alkylaryl, followed by replacement of that alkylaryl protecting group with an electron-withdrawing protecting group R² as hereinbefore defined.

The cyclisation process is preferably carried out under one or more of the following conditions.
(a) In the presence of a suitable solvent system. Suitable solvents include aromatic hydrocarbons (e.g. toluene), aliphatic hydrocarbons (e.g. cyclohexane) and halogenated (e.g. chlorinated) hydrocarbons such as chloroform and, particularly, dichloromethane.
(b) In the presence of a suitable catalyst (e.g. an acidic catalyst such as a Lewis acid or a Brönsted acid (e.g. a sulfonic acid such asp-toluenesulfonic acid)).
(c) At or above room temperature (e.g. from room temperature to the reflux temperature of the solvent system that is employed). When the solvent that is employed is dichloromehane, the reaction is preferably carried out at reflux.

The cyclisation process is preferably carried out to provide compounds in which R^{1a} and R^{1b}, together with the nitrogen atom to which they are attached, represent a cyclic imide such as a phthalimide group.

Further, the cyclisation process is preferably carried out on compounds of formula VI in which R^{2a} represents an alkylaryl group, such as benzyl, followed by replacement of that alkylaryl group with an electron-withdrawing amino protecting group by analogy with methods known to those skilled in the art (e.g. by a deprotection/protection procedure, which is optionally carried out in one step). For example, compounds of formula IV in which R² represents benzyloxycarbonyl may be prepared by cyclisation of a corresponding compound of formula VI, in which R^{2a} represents an alkylaryl group followed by reaction of the resultant intermediate with benzylchloroformate, for example as described hereinafter. Compounds of formula VI may be prepared by methods known to those skilled in the art. For example, compounds of formula VI may be prepared by reaction of a compound of formula VII, 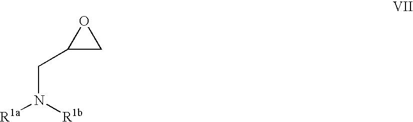
wherein R^{1a} and R^{1b} are as hereinbefore defined, with a compound of formula VIII, 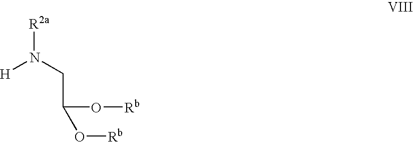
wherein R^{2a} and R^{2b} are as hereinbefore defined.

Preferred values of R^{1a, R}^{1b}, R^{2a}, and R^{b} include those mentioned hereinbefore.

Reaction of compounds of formula VII with compounds of formula VIII may be carried out under one or more of the following conditions:
(a) In the presence of a suitable solvent system. Suitable solvents include polar molecules (e.g. a hydroxylic solvents such as ethanol, methanol, propan-2-ol, or mixtures thereof (such as industrial methylated spirit), DMSO, acetonitrile, DMF etc.).
(b) At or above room temperature (e.g. from room temperature to the reflux temperature of the solvent system that is employed). When the solvent system that is employed is industrial methylated spirit, reaction is preferably carried out at reflux.
c) Under an appropriate inert atmosphere (e.g. under nitrogen).
(d) Using a molar ratio of the compound of formula VII to the compound of formula VIII of between 3:2 and 2:3 (e.g. between 11:10 and 10:11, such as 1:1).

Compounds of formula VI may, following their formation from compounds of formula VII and VIII, and without being isolated, be converted directly (i.e. in a “one-pot” procedure) to compounds of formula IV. This conversion may be achieved by addition of a suitable catalyst for the cyclisation reaction (e.g. an acidic catalyst such asp-toluenesulfonic acid) and/or by solvent exchange (e.g. from industrial methylated spirits to toluene or dichloromethane). Once formed, compounds of formula I may be converted to other compounds of formula I (e.g. by conversion of one R¹ and/or R² group to another) or to other compounds containing the oxabispidine ring system.

Thus, there is further provided a process for the preparation of a compound of formula IX, 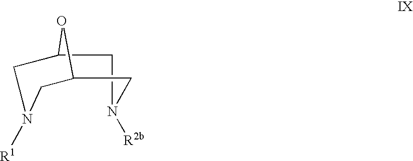
wherein R^{2b} represents H or R² and R¹ and R² are as hereinbefore defined, which process comprises reduction of a corresponding compound formula I, as hereinbefore defined, in the presence of a suitable reducing agent.

It is preferred that this reduction is carried out to produce compounds of formula IX in which R^{2b} is H. Thus, the reduction is preferably carried out using a compound of formula I in which R² represents an electron-withdrawing amino protecting group that may be cleaved under reducing conditions (e.g. the benzyloxycarbonyl group).

Preferred values of R¹ include those mentioned hereinbefore.

Suitable reducing agents include DIBALH or one or more hydrogenation catalysts in the presence of hydrogen. Suitable hydrogenation catalysts are known to those skilled in the art and include supported metal catalysts such as Pt/C, Rh/C and, particularly, Pd/C.

When the reducing agent is a hydrogenation catalyst in the presence of hydrogen, the formation of compounds of formula IX may be carried out under one or more of the following conditions:
(a) In the presence of a suitable solvent system. Suitable solvents include those comprising polar molecules (e.g. acetonitrile or hydroxylic compounds such as ethanol or, particularly, methanol, or mixtures thereof).
(b) At or above room temperature (e.g. from room temperature to 100° C.). When the solvent system that is employed is methanol, reaction is preferably carried out at room (i.e. ambient) temperature.
(c) At or above atmospheric pressure (e.g. between 100 and 400 kPa (1 to 4 bar), such as at 200 kPa).

Compounds of formulae III, V, VII and VIII, and derivatives thereof, are either commercially available, are known in the literature or may be obtained by analogy with the processes described herein, or by conventional synthetic procedures, in accordance with standard techniques, from readily available starting materials using appropriate reagents and reaction conditions. For example, compounds of formula VIII may be prepared according to, or by analogy with, the methods disclosed in Chem. Pharm. Bull. 40(2), 343 (1992).

The skilled person will appreciate that certain compounds of formulae I, II and IX may be prepared from certain other compounds of formulae I, II and IX, respectively, or from structurally related compounds.

In particular, compounds of formulae I, II or IX in which R¹ represents certain values of the structural fragment of formula Ia may be prepared from other compounds of formulae I, II or IX, 20 respectively, according to or by analogy with relevant processes known in the art for the synthesis or interconversion of compounds containing corresponding structural fragments of formula Ia. Such processes are described in, for example, international patent applications WO 99/31100, WO 00/76997, WO 00/76998, WO 00/76999, WO 00/77000 and, particularly, WO 01/28992.

Furthermore compounds of formula XI comprising amino protecting groups at one or both of the bispidine nitrogens may be deprotected under standard conditions, simultaneously and/or sequentially, and subsequently reacted with reagents to form compounds as described generically and specifically in WO 01/28992. Particular compounds that may be mentioned in WO 01/28992 include:
4-({3-[7-(3,3-dimethyl-2-oxobutyl)-9-oxa-3,7diazabicyclo[3.3.1]non-3-yl]propyl}amino)benzonitrile; tert-butyl 2-{7-[3-(cyanoanilino)propyl]-9-oxa-3,7-diazabicyclo[3.3.1]non-3-yl}ethylcarbamate; tert-butyl 2-{7-[4(4-cyanophenyl)butyl]-9-oxa-3,7-diazabicyclo[3.3.1]non-3-yl}ethylcarbamate; and tert-butyl 2-{7-[(2S)-3-(4-cyanophenoxy)-2-hydroxypropyl]-9-oxa-3,7-diazabicyclo[3.3.1]non-3-yl}ethylcarbamate (the compounds of Examples 3, 7, 8 and 9, respectively, of that document), which compounds may be prepared from compounds of formula XI (e.g. a compound of formula XI in which R¹ represents benzyl and R² represents H; 3-benzyl-9-oxa-3,7-diazabicyclo[3.3.1]nonane; see Preparations A(iii) and N(iv) of WO 01/28992), in accordance with the processes described in WO 01/28992, the relevant disclosures of which document (e.g. Preparations A, B, C, G and N and Examples 3, 7, 8 and 9) are hereby incorporated by reference.

It will be appreciated by those skilled in the art that, in the processes described above, the functional groups of intermediate compounds may be, or may need to be, protected by protecting groups. Functional groups which it is desirable to protect include hydroxy and amino. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include the amino protecting groups mentioned hereinbefore, such as benzyl, sulfonyl (e.g. benzenesulfonyl), tert-butyloxycarbonyl, 9-fluorenylmethoxycarbonyl or benzyloxycarbonyl.

The protection and deprotection of functional groups may take place before or after any of the reaction steps described hereinbefore.

Protecting groups may be removed in accordance with techniques that are well known to those skilled in the art and as described hereinafter.

The use of protecting groups is fully described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

Those skilled in the art will appreciate that the processes described herein may be used to prepare compounds of formulae I, II, IV, VI and IX in any given stereochemical form.

The processes described herein may be utilised in the synthesis of compounds comprising the oxabispidine moiety. The processes possess the surprising advantage that compounds of formula I (as well as compounds of formulae II, IV, VI and IX) may be prepared in higher yields, in less time, more conveniently, and at a lower cost, than when prepared according to the process described in the prior art.

In particular, processes described herein may have the advantage that a lower proportion of the monocyclic precursors of compounds of formula I (i.e., in the present case, the compound of formula II) is incapable of forming oxabispidine product as compared to the precursors employed in the processes described in WO 01/28992. In this manner, the process of the invention may have the advantage that the production of compounds of formulae I and IX involves less wastage of unreactive intermediate products than the production of the same or similar compounds according to processes described in WO 01/28992.

Further, the elimination process described herein has the advantage that it offers particularly in conjunction with the previous steps of reaction of a compound of formula VII with a compound of formula VIII, and the cyclisation process as hereinbefore described) a concise synthesis for the formation of 2,3-dihydrooxazines (such as 2-aminomethyl-substituted 2,3-dihydrooxazines), which compounds are useful in the synthesis of the oxabispidine ring system.

Moreover, the processes described herein have the advantage that oxabispidine compounds may be produced without the use of protecting groups that may have disadvantageous properties.

The invention is illustrated, but in no way limited, by the following examples.

### EXAMPLES

General Experimental Procedures

Mass spectra were recorded on one of the following instruments: a Waters ZMD single quad with electrospray (S/N mc350); a Perkin-Elmer SciX API 150ex spectrometer; a VG Quattro II triple quadrupole; a VG Platform II single quadrupole; or a Micromass Platform LCZ single quadrupole mass spectrometer (the latter three instruments were equipped with a pneumatically assisted electrospray interface (LC-MS)). ¹H NMR and ¹³C NMR measurements were performed on Varian 300, 400 and 500 spectrometers, operating at ¹H frequencies of 300, 400 and 500 M respectively, and at ¹³C frequencies of 75.5, 100.6 and 125.7 MHz respectively.

Rotamers may or may not be denoted in spectra depending upon ease of interpretation of spectra Unless otherwise stated, chemical shifts are given in ppm with the solvent as internal standard.

### Example 1

### N-{3-[N′-(2,2-Dimethoxyethyl)-N′-benzyl]amino-2-hydroxypropyl}-phthalimide

N-(Oxiranylmethyl)phthalimide (13.6 g, 0.067 mol, 1 eq., Fluka) was dissolved in 260 mL (60 vols) of IMS, in a 500 mL 3-neck flask fitted with a condenser, under a nitrogen atmosphere. N-Benzylaminoacetaldehyde dimethyl acetal (13 g, 0.067 mol, 1 eq.; see, for example, Chem. Pharm. Bull. 40(2), 343 (1992)) was then added to this solution. The solution was then heated to reflux for twenty hours. The reaction was then allowed cool to ambient temperature, and the solvent removed under vacuum to yield the title compound as a yellow oil. Yield=26.25 g (99%).

C₂₂H₂₆N₂O₅

LC/MS: 399 (M⁺)

### Example 2

### N-[(4-Benzyl-6-methoxymorpholin-2-yl)methyl]phthalimide

N-{3-[N′-(2,2-Dimethoxyethyl)-N′-benzyl]amino-2-hydroxypropyl}-phthalimide (25.5 g, 0.064 mol, 1 eq.; see Example 1 above) was dissolved in dichloromethane (275 mL, 11 vols) in a 500 mL 3-neck flask, fitted with a condenser, under a nitrogen atmosphere to yield a yellow solution.

p-Toluenesulfonic acid (1.25 g, 6.4 mmol, 0.1 eq.) was then added to this solution and the reaction heated to reflux for eighteen hours. The reaction was allowed to cool, and was then washed with 75 mL of 1 M NaHCO₃, followed by 75 mL water. The organic layer was dried over MgSO₄, and the solvent removed under vacuum to yield the title compound as an orange oil. Yield=22.7 g, (97%).

C₂₁H₂₂N₂O₄

LC/MS: 367 (M⁺)

### Example 3

### N-[(4-Benzyloxycarbonyl-6-methoxymorpholin-2-yl)methyl]phthalimide

N-[(4-Benzyl-6-methoxymorpholin-2-yl)methyl]phthalimide (15 g, 0.041 mol, 1 eq.; see Example 2 above) was dissolved in dichloromethane (15 mol, 10 vols) under nitrogen yielding an orange solution. Benzyl chloroformate (15.4 mL, 0.045 mol, 1.1 eq. 50% solution in toluene) was then added and the reaction allowed to stir at ambient temperature over two days. The reaction was then diluted using 225 mL dichloromethane, and washed with NaOH (1 M, 375 mL), then water (375 mL). The organic layer was dried over MgSO₄ and concentrated to a dark orange oil. This crude product was purified using a Flash 75 Biotage™ column, eluting from a 3:1 iso-hexane/ethyl acetate gradient to 1:3, using 17 L of solvent in total. The fractions that contained product were concentrated under vacuum to yield the title compound as an orange oil. Yield=12.8 g (76%).

C₂₂H₂₂N₂O₆

LC/MS: 411.1 (M³⁰ )

### Example 4

### N-[(4-Benzyloxycarbonyl-2,3-dihydrooxazin-2-yl)methyl]phthalimide

N-[(4-Benzyloxycarbonyl-6-methoxymorpholin-2-yl)methyl]phthalimide (12.3 g, 0.03 mol, 1 eq; see Example 3 above) was dissolved in toluene (250 mL, 20 vols) in a 500 mL 3-necked flask under nitrogen. The flask was then fitted with a condenser and soxhlet extractor containing 3 Å molecular sieves. p-Toluenesulfonic acid (0.58 g, 3 mmol, 0.1 eq.) was added to the solution, and the reaction was heated to reflux for eight hours. Analysis after this period showed that the reaction had not gone to completion. A further 0.1 eq. (0.58 g, 3 mmol, 0.1 eq.) of p-toluenesulfonic acid was added. After a further four hours at reflux the reaction was allowed to cool. The reaction mixture was then poured into saturated NaHCO₃ (aq) and separated. The aqueous layer was then washed with 2×250 mL dichloromethane. All the organic extracts were then combined, dried over MgSO₄, and concentrated under vacuum to an oil. This crude product was purified using a Flash 75 Biotage™ column, eluting from a 3:1 iso-hexane/ethyl acetate solvent system, to 7:3, using 10 L of solvent. The fractions that contained product were combined and concentrated to a colourless oil, which crystallised upon standing to give the title compound as a colourless solid. Yield=7.4 g (65%).

C₂₁H₁₈N₂O₆

LC/MS: 379 (M⁺)

Melting point 96° C.

¹H NMR (299.946 MHz, d₆-DMSO): δ 7.92-7.84 (m), 7.43-7.34 (m), 6.25-6.21 (m), 6.03 (dd, J=32.1, 4.8 Hz), 5.15 (d, J=4.0 Hz), 4.31-4.15 (m), 4.00-3.76 (m), 3.33-3.23 (m).

### Example 5

### 2-Aminomethyl-4-benzyloxycarbonyl-2,3-dihydrooxazine

N-[(4Benzyloxycarbonyl-2,3dihydrooxazin-2-yl)methyl]phthalimide (7.2 g, 0.019 mol; see Example 4 above) was dissolved in a solution of hydrazine (72 mL, 10 vol, 1 M solution in THF) and stirred at ambient temperature for ten hours, forming a slurry of a white precipitate. The slurry was filtered, and the filtrate concentrated under vacuum, yielding an off-white solid. This solid was slurried in 50 mL ethyl acetate, and then filtered to yield the title compound as a colourless, crystalline solid. Yield=3.66 g (93%).

C₁₃H₁₆N₂O₃

LC/MS: 249 (M⁺)

Melting point 101° C.

¹H NMR (299.944 MHz, CDCl₃): δ 7.36-7.33 (m), 6.30 (dd, J=39.1, 4.3 Hz), 5.96 (dd, J=37.7,4.4 Hz), 5.18 (s), 4.04-3.92 (m), 3.33-3.20 (m), 2.97 (d, J=6.0 Hz)

### Example 6

### 2-(N-Benzylamino)methyl-4-benzyloxycarbonyl-2,3-dihydrooxazine

2-Aminomethyl-4-benzyloxycarbonyl-2,3-dihydrooxazine (3.5 g, 14.1 mmol, see Example 5 above) was suspended in methanol (35 mL) and heated to 50° C. Benzaldehyde (1.43 mL, 14.0 mmol) was added at this temperature. The mixture was heated at reflux for 30 min, allowed to cool to ambient temperature and stirred overnight. The absence of starting material was confirmed by ¹H NMR spectroscopy. The mixture was heated to 50° C., and a solution of sodium borohydride (0.8 g, 21.0 mmol) in methanol (15 mL) was added over a period of 15 min. The reaction was determined to be incomplete by HPLC, so further sodium borohydride (0.8 g, 21.0 mmol) was added. After 1 hour the reaction was deemed to be complete by HPLC. The mixture was allowed to cool to ambient temperature and water (40 mL) was added. The methanol was removed by evaporation under reduced pressure, and ethyl acetate (150 mL) and water (100 mL) were added. The organic layer was separated and washed with water (100 mL) and brine (50 mL). The combined aqueous layers were washed with ethyl acetate (100 mL). The organic washings were combined, dried with sodium sulphate and evaporated under reduced pressure to give a yellow oil. This crude product was purified by flash column chromatography, eluting from 90:9:1 to 50:49:1 iso-hexane:ethyl acetate:triethylamine, using 3 L of solvent. The product-containing fractions were evaporated under reduced pressure to give the title compound (0.68 g, 2.0 mm ol, 14%) as a colourless oil.

LC-MS: 337(M⁺)

### Example 7

### Benzyl 7-benzyl-2-methoxy-9-oxa-3,7-diazabicyclo[3.3.1]nonane-3-carboxylate

2-(N-Benzylamino)methyl-4-benzyloxycarbonyl-2,3-dihydrooxazine (0.5 g, 1.5 mmol, 1 eq.; see Example 6 above) was dissolved in acetonitrile,

(10 mL, 20 vols) under nitrogen, yielding a colourless solution. To this solution, dimethoxymethane (0.4 mL, 4.4 mmol, 3 eq.) and p-toluene-sulfonic acid (0.03 g, 0.13 mmol, 0.1 eq.) were added. The reaction was then heated to reflux. After two hours at reflux, no reaction was observed, hence the reaction was allowed to cool. Once at ambient temperature, paraformaldehyde (0.54 g, 4.4 mmol, 3 eq.), and methanol (5 mL, 10 vols) were added to form a slurry. The reaction was then refluxed for one hour, after which time no starting material remained (as determined by HPLC analysis). The reaction was then cooled, the paraformaldehyde filtered off and the filtrate concentrated to an oil under vacuum. The oil product was then dissolved in ethyl acetate (100 mL), and washed with aqueous sodium bicarbonate (2×100 mL). The aqueous extracts were washed with ethyl acetate (100 mL) and the organic washes were then combined, dried over Na₂SO₄ and concentrated to give a yellow oil. This crude product was then purified by column chromatography, eluting with iso-hexane/ethyl acetate (3:1) using 1 L of solvent. The fractions that contained product were combined and concentrated to give the title compound as a colourless oil. Yield=0.4 g (71%).

C₂₂H₂₆N₂O₄

LC/MS: 383(M⁺)

### Example 8

### 3-Benzyl-9-oxa-3,7-diazabicyclo[3.3.1]nonane

Benzyl 7-benzyl-2-methoxy-9-oxa-3,7-diazabicyclo[3.3.1]nonane-3-carboxylate (200 mg, 0.53 mmol; see Example 7 above) was dissolved in methanol (3 mL, 15 vols). Pd/C (100 mg, Johnson Matthey catalyst type 87L) was then added to this solution washed in with 1 mL methanol. The reaction was then stirred under a hydrogen atmosphere, at 2 bar pressure and ambient temperature for two hours. The reaction was removed from the hydrogen atmosphere, and filtered through Celite®, removing palladium catalyst The filtrate was then concentrated to yield the title compound as a colourless oil. Yield=110 mg (96%) C₁₃H₁₈N₂O

LC/MS: 219 (M⁺)

¹H NMR (300 MHz, D₂O): δ 7.50 (5H, s), 4.06 (2H, br s), 3.91 (2H, br s), 3.50-3.61 (4H,m), 3.39 (2H, d) and 3.08 (2H, br s)

Abbreviations

| | |
|---|---|
| API | atmospheric pressure ionisation (in relation to MS) |
| br | broad (in relation to NMR) |
| d | doublet (in relation to NMR) |
| dd | doublet of doublets (in relation to NMR) |
| Et | ethyl |
| eq. | equivalents |
| h | hour(s) |
| HPLC | high performance liquid chromatography |
| IMS | industrial methylated spirit |
| m | multiplet (in relation to NMR) |
| Me | methyl |
| min. | minute(s) |
| MS | mass spectroscopy |
| Pd/C | palladium on carbon |
| q | quartet (in relation to NMR) |
| rt | room temperature |
| s | singlet (in relation to NMR) |
| t | triplet (in relation to NMR) |

Prefixes n-, s-, i-, t- and tert- have their usual meanings: normal, secondary, iso, and tertiary.

1. A process for the preparation of a compound of formula I, 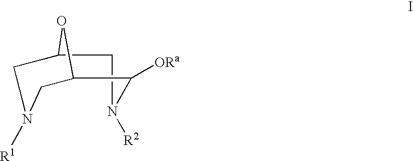
wherein
R¹ represents H, aryl or a structural fragment of formula Ia, 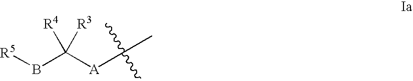
in which
R³ represents H, halo, C₁₋₆ alkyl, —OR⁶, -E-N(R⁷)R⁸ or, together with R⁴, represents ═O;
R⁴ represents H, C₁₋₆ alkyl or, together with R³, represents ═O;
R⁶ represents H, C₁₋₆ alkyl, -E-aryl, -E-Het¹, —C(O)R^{9a}, C(O)OR^{9b} or —C(O)N(R^{10a})R^{10b};
R⁷ represents H, C₁₋₆ alkyl, -E-aryl, -E-Het¹, —C(O)R^{9a}, C(O)OR^{9b},
S(O)₂R^{9c}, [C(O)]ₚN(R^{10a})R^{10b} or —C(NH)NH₂;
R⁸ represents H, C₁₋₆ alkyl, -E-aryl or —C(O)R^{9d};
R^{9a} to R^{9d} independently represent, at each occurrence,
C₁₋₆ alkyl (optionally substituted and/or terminated by one or more substituents selected from halo, aryl and Het²), aryl, Het³, or R^{9a} and R^{9d} independently represent H;
R^{10a} and R^{10b} independently represent, at each occurrence, H or C₁₋₆ alkyl (optionally substituted and/or terminated by one or more substituents selected from halo, aryl and Het⁴), aryl, Het⁵, or together represent C₃-6 alkylene, optionally interrupted by an O atom; E represents, at each occurrence, a direct bond or
C₁₋₄ alkylene;
p represents 1 or 2;
A represents -G-, -J-N(R¹¹)— or -J-O— (in which latter two groups, N(R¹¹)— or O— is attached to the carbon atom bearing R³ and R⁴);
B represents -Z-, -Z-N(R¹²)—, —N(R¹²)-Z-, -Z-S(O)ₙ— or -Z-O— (in which latter two groups, Z is attached to the carbon atom bearing R³ and R⁴);
G represents a direct bond or C₁₋₆ alkylene;
J represents C₂₋₆ alkylene;
Z represents a direct bond or C₁₋₄ alkylene;
R¹¹ and R¹² independently represent H or C₁₋₆ alkyl;
n represents 0, 1 or 2;
R⁵ represents aryl or heteroaryl, both of which groups are optionally substituted by one or more substituents selected from —OH, cyano, halo, nitro, C₁₋₆ alkyl (optionally terminated by —N(H)C(O)OR^{13a}), C₁₋₆ alkoxy,
—N(R^{14a})R^{14b}, —C(O)R^{14c}, —C(O)OR^{14d}, —C(O)N(R^{14e})R^{14f}, —N(R^{14g})C(O)R^{14h}, —N(R¹⁴ⁱ)C(O)N(R^{14j})R^{14k}, —N(R^{14m})S(O)₂R^{13b}, —S(O)₂R^{13c} and/or —OS(O)₂R^{13d};
R^{13a} to R^{13d} independently represent C₁₋₆ alkyl;
R^{14a} and R^{14b} independently represent H, C₁₋₆ alkyl or together represent C₃₋₆ alkylene, resulting in a four- to seven-membered nitrogen-containing ring;
R^{14c} to R^{14m} independently represent H or C₁₋₆ alkyl;
Het¹ to Het⁵ independently represent, at each occurrence, five- to twelve-membered heteroaryl groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, N(R^{15a})R^{15b}, —C(O)R^{15c}, —C(O)OR^{15d}, —C(O)N(R^{15c})R^{15f}, —N(R^{15g})C(O)R^{15h} and —N(R¹⁵ⁱ)S(O)₂R^{15j};
R^{15a} to R^{15j} independently represent C₁₋₆alkyl, aryl or R^{15a} to R^{15j} independently represent H; and
wherein each aryl and aryloxy group, unless otherwise specified, is optionally substituted; provided that:
(a) when R⁴ represents H or C₁₋₄ alkyl; and
A represents -J-N(R¹¹) or -J-O—;
then B does not represent —N(R¹²)—, —S(O)ₙ—, —O— or —N(R¹²)-Z- (in which latter group —N(R¹²) is attached to the carbon atom bearing R³ and R⁴);
(b) when R³ represents —OR⁶ or -E-N(R⁷)R⁸ in which E represents a direct bond, then:
(i) A does not represent a direct bond, -J-N(R¹¹)— or -J-O—; and
(ii) B does not represent —N(R¹²)—, —S(O)ₙ—, —O— or —N(R¹²)-Z- (in which latter group —N(R¹²) is attached to the carbon atom bearing R³ and R⁴);
(c) when A represents a direct bond, then R³ and R⁴ do not together represent ═O;
R² represents an electron withdrawing amino protecting group; and
R^{a} represents C₁₋₄ alkyl or benzyl,
which process comprises reaction of a compound of formula II, 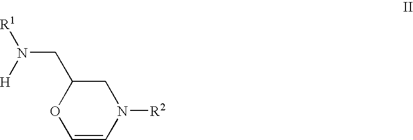
wherein R¹ and R² are as defined above, with either:
(a) a formaldehyde and a compound of formula III,
R^{a}—OH III
wherein R^{a} is as defined above; and/or
(b) a protected derivative of a formaldehyde.

2. A process as claimed in claim 1, wherein R² is a benzyloxycarbonyl group.

3. A process as claimed in claim 1, wherein the formaldehyde is paraformaldehyde.

4. A process as claimed in claim 1 wherein R¹ represents a structural fragment of formula Ia.

5. A process as claimed in claim 1 wherein R³ represents H or —OH.

6. A process as claimed in claim 1 wherein R⁴ represents H.

7. A process as claimed in claim 1 wherein A represents a direct bond or methylene.

8. A process as claimed in claim 1 wherein B represents -Z-, -Z- N(H)— or -Z-O—.

9. A process as claimed in claim 8, wherein B represents a single bond.

10. A process as claimed in claim 1 wherein R⁵ represents phenyl, which latter group is optionally substituted by cyano.

11. A process as claimed in claim 10, wherein R⁵ represents unsubstituted phenyl.

12. A process as claimed in claim 1 wherein R¹ represents benzyl.

13. A process as claimed in claim 1 wherein R^{a} represents methyl.

14. A process as claimed in claim 1 wherein the reaction is carried out in the presence of acetonitrile, a C₁₋₄ alkyl alcohol, toluene or a mixture thereof.

15. A process as claimed in claim 1 wherein the reaction is carried out in the presence of p-toluenesulfonic acid.

16. A process as claimed in claim 1, wherein the reaction is carried out at reflux temperature.

17. A process as claimed in claim 1 wherein the reaction is carried out using one or more equivalents (relative to the compound of formula II) of the formaldehyde.

18. A process as claimed in claim 1 wherein the reaction is carried out using one or more equivalents (relative to the compound of formula II) of the compound of formula III.

19. A process as claimed in claim 1 wherein the reaction is carried out by reacting the compound of formula II with one or more equivalents of the formaldehyde, in the presence of an excess of a compound of formula III.

20. A process as claimed in claim 1 wherein the compound of formula II is prepared by elimination of R^{b}OH from a compound of formula IV, 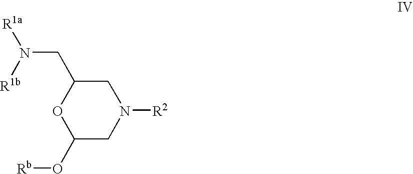
wherein
R^{1a} represents an aryl group, a structural fragment of formula Ia as hereinbefore defined, an electron withdrawing amino protecting group as hereinbefore defined, or, together with R^{1b}, represents a cyclic amino protecting group;
R^{1b} represents an electron withdrawing amino protecting group as hereinbefore defined, or, together with R^{1a}, represents a cyclic amino protecting group;
R^{b} represents C₁₋₄ alkyl; and
R² is as defined in claim 1,
followed by deprotection (as necessary) of the nitrogen atom to which the groups R^{1a} and R^{1b} are attached.

21. A process for the formation of a compound of formula II as defined in claim 1, which process comprises elimination of R^{b}OH from a compound of formula IV, 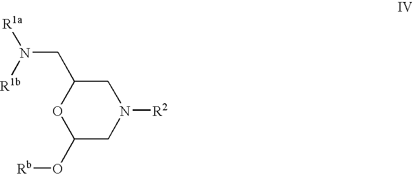
wherein
R^{1a} represents an aryl group, a structural fragment of formula Ia as hereinbefore defined, an electron withdrawing amino protecting group as hereinbefore defined, or, together with R^{1b}, represents a cyclic amino protecting group;
R^{1b} represents an electron withdrawing amino protecting group as hereinbefore defined, or, together with R^{1a}, represents a cyclic amino protecting group;
R^{b} represents C₁₋₄ alkyl; and
R² is as defined in claim 1,
followed by deprotection (as necessary) of the nitrogen atom to which the groups R^{1a} and R^{1b} are attached.

22. A process as claimed in claim 20, wherein the elimination is carried out on a compound of formula IV in which R^{1a} and R^{1b} together represent a cyclic amino protecting group.

23. A process as claimed in claim 20, wherein the cyclic amino protecting group forms a phthalimide group with the nitrogen atom to which R^{1a} and R^{1b} are attached.

24. A process as claimed in claim 20, wherein R² represents benzyloxycarbonyl.

25. A process as claimed in claim 20, wherein R^{b} represents methyl.

26. A process as claimed in claim 20, wherein the elimination process is carried out in the presence of toluene.

27. A process as claimed in claim 20, wherein the elimination process is carried out in the presence of p-toluenesulfonic acid.

28. A process as claimed in claim 20, wherein the elimination process is carried out at elevated temperature.

29. A process as claimed in claim 20, wherein the elimination process is carried out in the presence of an alcohol sorbing agent.

30. A process as claimed in claim 20, wherein R^{1a} and R^{1b} together represent a cyclic amino protecting group, and the deprotection is carried out by way of reaction with hydrazine.

31. A process as claimed in claim 20, wherein the deprotection is thereafter followed by reaction of the deprotected amine with a compound that provides the aryl group or the structural fragment of formula Ia.

32. A process as claimed in claim 31, wherein the reaction is carried out by reaction of a compound of formula II in which R¹ represents H with a compound of formula V,
R⁵BC(R³)(R⁴)AL¹ V
wherein L¹ represents a suitable leaving group and A, B, R³, R⁴ and R⁵ are as defined in claim 1.

33. A process as claimed in claim 31, wherein the reaction is carried out to provide a compound of formula II in which R¹ is benzyl by reaction of a compound of formula II in which R¹ represents H with benzaldehyde followed by reduction of the resultant intermediate.

34. A process as claimed in claim 20 wherein the compound of formula IV is prepared by cyclisation of a compound of formula VI, 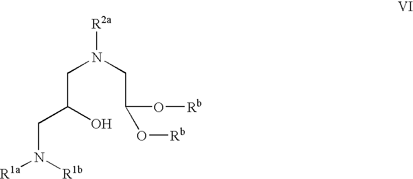
wherein
R^{2a} represents an amino protecting group; and
R^{1a}, R^{1b} and R^{b} are as defined in claim 20.

35. A process for the formation of a compound of formula IV as defined in claim 20, which process comprises cyclisation of a compound of formula VI, 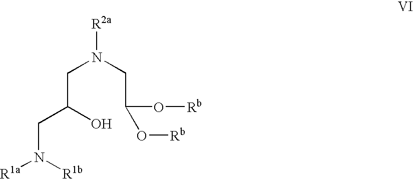
wherein
R^{2a} represents an amino protecting group; and
R^{1a}, R^{1b} band R^{b} are as defined in claim 20.

36. A process as claimed in claim 34, wherein R^{2a} represents C₁₋₃ alkylphenyl.

37. A process as claimed in claim 36, wherein R^{2a} represents benzyl.

38. A process as claimed in claim 34, wherein the cyclisation process is followed by replacement of the group R^{2a} with a group R² as defined in claim 1.

39. A process as claimed in claim 38, wherein the group R² represents benzyloxycarbonyl.

40. A process as claimed in claim 34 wherein the compound of formula VI is prepared by reaction of a compound of formula VII, 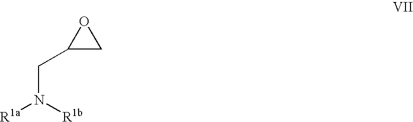
wherein R^{1a} and R^{1b} are as defined in claim 20, with a compound of formula VIII, 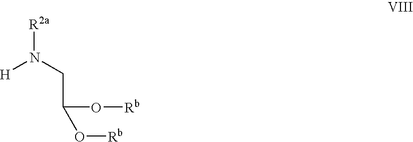
wherein R^{2a} is as defined in claim 34 and R^{b} is as defined in claim 20.

41. A process for the formation of a compound of formula VI as defined in claim 34, which process comprises reaction of a compound of formula VII, 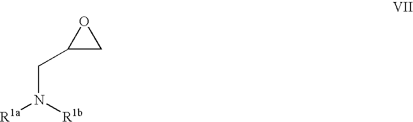
wherein R^{1a} and R^{1b} are as defined in claim 20, with a compound of formula VIII, 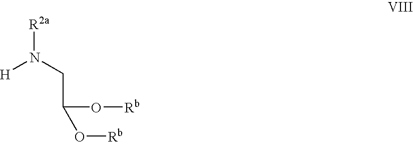
wherein R^{2a} is as defined in claim 34 and R^{b} is as defined in claim 20.

42. A process for the preparation of a compound of formula IX, 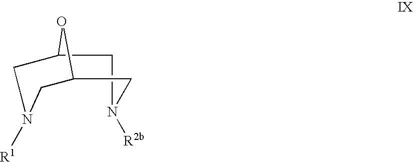
wherein R^{2b} represents H or R² and R¹ and R² are as defined in claim 1, which process comprises preparation of a compound of formula I as claimed in claim 1, followed by reduction the compound of formula I so formed.

43. A process for the preparation of a compound of formula IX, 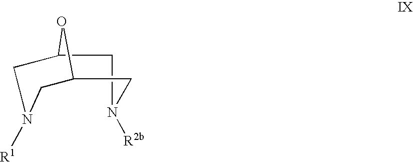
wherein R^{2b} represents H or R² and R¹ and R² are as defined in claim 1, which process comprises reduction a compound of formula I as defined in claim 1.

44. A process as claimed in claim 42 wherein, in the compound of formula IX, R^{1b} is H.

45. A process as claimed in claim 42, wherein R¹ represents benzyl.

46. A process as claimed in claim 42, wherein the reduction is carried out in the presence of a hydrogenation catalyst in the presence of hydrogen.

47. A process as claimed in claim 42, wherein either or both of the groups R¹ and (if present) R^{2b} are removed, simultaneously and/or sequentially, and the resultant compound is subsequently reacted with reagents to form any one of:
4-({3-[7-(3,3-dimethyl-2-oxobutyl)-9-oxa-3,7-diazabicyclo[3.3.1]non-3-yl]propyl}amino)benzonitrile;
tert-butyl 2-{7-[3-(4-cyanoanilino)propyl]-9-oxa-3,7-diazabicyclo [3.3.1]-non-3-yl}ethylcarbamate;
tert-butyl 2-{7-[4-(4-cyanophenyl)butyl]-9-oxa-3,7-diazabicyclo[3.3.1]non-3-yl}ethylcarbamate; or
tert-butyl 2-{7-[(2S)-3-(4-cyanophenoxy)-2-hydroxypropyl]-9-oxa-3,7-di-azabicyclo[3.3.1]non-3-yl}ethylcarbamate.

48. A compound of formula II as defined in claim 1 or a protected derivative thereof.

49. A compound of formula IV as defined in claim 20 or a protected derivative thereof.

50. A compound of formula VI as defined in claim 34 or a protected derivative thereof.
